(19) United States
(12) Patent Application Publication
(10) Pub. No.: US 2005/0148502 A1
(43) Pub. Date: Jul. 7, 2005

(54) Compositions and methods for preventing and treating cancer via modulating ube1l, isg15 and/or ubp43

(76) Inventors: Ethan Dmitrovsky, Hanover, NH (US); Bret A Hassel, Woodbine, MD (US); Sutisak Kitareewan, Ordford, NH (US); Ian Pitha-Rowe, White River Junction, NH (US)

Correspondence Address: LICATLA & TYRRELL P.C., 66 E. MAIN STREET, MARLTON, NJ, 08053, US

(21) Appl. No.: 10/506,226

(22) PCT Filed: Mar. 5, 2003
(86) PCT No.: PCT/US03/06905

Related U.S. Application Data
(60) Provisional application No. 60/361,830, filed on Mar. 5, 2002.

(51) Int. Cl. A61K 38/48; A61K 38/17

(52) U.S. Cl. 514/12; 424/94.63

(57) ABSTRACT

Compositions and methods of using compositions that induce UBE1L or a ubiquitin-like protein ISG15 or inhibit a deconjugase UBP43 to degrade oncogenic proteins and enhance apoptosis of cancer (neoplastic) or pre-cancerous (pre-neoplastic) cells are provided. Methods for the prevention or treatment of cancer via administration of these compositions are also provided.

### INTRODUCTION

This work was supported in part by National Institutes of Health Grant RO-1-CA62275 and National Institutes of Health Grant RO-1-CA87546 and the U.S. Government may have certain rights in this invention.

### FIELD OF THE INVENTION

UBE1L, the ubiquitin-like protein ISG15 and the deconjugase UBP43 have now been identified as direct pharmaceutical targets that overcome oncogenic effects of oncogenic proteins such as PML/RARα, triggering degradation of the oncogenic proteins and signaling apoptosis-in cancer cells expressing the oncogenic proteins. The present invention relates to new compositions as well as methods of designing compositions targeted to UBE1L and/or ubiquitin-like proteins such as ISG15 or the deconjugase UBP43 which are useful in enhancing the pro-apoptotic and degradative pathway that opposes effects of oncogenic proteins such as PML/RARα. The present invention also relates to methods for preventing and treating cancer which involve inducing UBE1L and/or ubiquitin-like proteins such as ISG15 or, alternatively, inhibiting the deconjugase UBP43 to increase programmed cell death (apoptosis) in neoplastic and pre-neoplastic cells.

### BACKGROUND OF THE INVENTION

Acute promyelocytic leukemia (APL) (FAB M3) cases express the oncogenic product of the t(15;17) chromosomal rearrangement, promyelocytic leukemia (PML)/retinoic acid receptorα (RARα) (Nason-Burchenal et al. (1996) in Molecular Biology of Cancer, ed. Bertino, J. R. (Academic San Diego), 1st Ed., pp 1547-1560; Nason-Burchenal, K. and Dmitrovsky, E. (1999) in Retinoids: The Biochemical and Molecular Basis of Vitamin A and Retinoid Action, eds. Nau, H. & Blaneer, W. (Springer, Berlin), pp. 301-322). All-trans-retinoic acid (RA) treatment causes complete remissions in these APL cases through induction of leukemic cell differentiation (Nason-Burchenal et al. (1996) in Molecular Biology of Cancer, ed. Bertino, J. R. (Academic San Diego), 1st Ed., pp 1547-1560; Nason-Burchenal, K. and Dmitrovsky, E. (1999) in Retinoids: The Biochemical and Molecular Basis of Vitamin A and Retinoid Action, eds. Nau, H. & Blaneer, W. (Springer, Berlin), pp. 301-322). A hallmark of RA response in APL is PML/RARα degradation that reverses PML/RARα oncogenic effects (Kakizuka et al. Cell 1991 68: 663-674; de Th6 et al. Cell 1991 68: 675-684; Yoshida et al. Cancer Res. 1996 56: 2945-2948; Raelson et al. Blood et al. 1996 88: 2826-2832; Nervi et al. Blood 1998 92: 2244-2251; Zhu et al. Proc. Natl. Acad. Sci USA 1999 96: 14807-14812). Proteasomal inhibitors prevent PML/RARα proteolysis, despite RA treatment, which is indicative of a proteasome-dependent pathway in this degradation (Yoshida et al. Cancer Res. 1996 56: 2945-2948; Raelson et al. Blood et al. 1996 88: 2826-2832; Nervi et al. Blood 1998 92: 2244-2251; Zhu et al. Proc. Natl. Acad. Sci USA 1999 96: 14807-14812). PML/RARα expression results in dominant-negative transcriptional repression (Kakizuka et al. Cell 1991 68: 663-674; de The et al. Cell 1991 68: 675-684). This repression is antagonized by pharmacological RA dosages that overcome inhibitory effects on transcription of the N-Cor/SMRT corepressor complex that has histone deacetylase activity (Lin et al. Nature (London) 1998 391: 811-814; Grignani et al. Nature (London) 1998 391: 815 818). RA treatment recruits a coactivator complex that stimulates transcription, resulting in activation of target genes (Lin et al. Nature (London) 1998 391: 811-814; Grignani et al. Nature (London) 1998 391: 815-818).

To understand the molecular basis of RA response in APL, identification of RA target genes is required.

GOS2 is a putative RA target gene identified by microarray analysis of APL cells (Tamayo et al. Proc. Natl. Acad. Sci. USA 1999 96: 2907-2912). The precise function of GOS2 is not yet known, but it was first identified as regulated during the cell cycle (Russell, L. and Forsdyke, D. R. DNA Cell Biol. 1991 10: 581-591), suggesting a role in cell cycle control.

Another candidate retinoid target gene is the CCAAT/enhancer binding protein ε (C/EBP ε) that contributes to retinoid transcriptional effects in APL (Park et al. J. Clin. Invest. 1999 103: 1399-1408). However, this species has not been linked to the degradation of PML/RARα.

Recent microarray analysis of RA-treated NB4 APL cells reported the prominent induction of UBE1L (ubiquitin-activating enzyme E1-like)(Tamayo et al. Proc. Natl. Acad. Sci. USA 1999 96: 2907-2912). The proteasome-dependent degradation of PML/RARα has also been proposed as a mechanism by which RA overcomes PML/RARα oncogenic effects (Yoshida et al. Cancer Res. 1996 56: 2945-2948; Raelson et al. Blood et al. 1996 88: 2826-2832; Nervi et al. Blood 1998 92: 2244-2251; Zhu et al. Proc. Natl. Acad. Sci USA 1999 96: 14807-14812). UBE1L could account for the proteasome dependent degradation of PML/RARα and perhaps other oncogenic proteins.

Hammerhead ribozymes that target PML/RARα have been used to show how PML/RARα degradation signals apoptosis but not differentiation in transfected APL cells that are either RA-sensitive or RA-resistant (Nason-Burchanel et al. Blood 1998 92: 1758-1767; Nason-Burchanel et al. Oncogene 1998 17: 1759-1768).

It has now been found that UBE1L is a retinoid target gene in APL that antagonizes PML/RARα oncogenic effects by triggering PML/RARα degradation. The consequence of this action is the promotion of apoptosis resulting in anti-oncogenic effects of UBE1L in APL and potentially in other neoplastic or pre-neoplastic cell contexts. Accordingly, compositions which target UBE1L or other proteins related thereto, such as the ubiquitin-like protein ISG15 or the deconjugase UBP43, are expected to be useful in preventing and treating cancer.

### SUMMARY OF THE INVENTION

An object of the present invention is to provide compositions for prevention and treatment of cancer comprising an agent which induces UBE1L and/or a ubiquitin-like protein such as ISG15 or an agent which inhibits the deconjugase UBP43.

Another object of the present invention is to provide a method for identifying agents as potential therapeutics against cancer which comprises determining the ability of the agent to induce UBE1L and/or ubiquitin proteins such as ISG15 or determining the ability of the agent to inhibit the deconjugase UBP43.

Another object of the present invention is to provide a method for enhancing pro-apoptotic and degradative pathways of neoplastic (cancerous) cells or pre-neoplastic (pre-cancerous) cells with an agent which induces UBE1L and/or ubiquitin-like proteins such as ISG15 or an agent which inhibits the deconjugase UBP43.

Another object of the present invention is to provide a method for preventing or treating cancer in a patient which comprises administering to a patient an agent which induces UBE1L and/or ubiquitin-like proteins such as ISG15 or an agent which inhibits UBP43.

### DETAILED DESCRIPTION OF THE INVENTION

The present invention relates to anticancer agents, methods for designing new anticancer agents and methods of using agents that induce activity and/or expression of ubiquitin-activating enzyme E1-like protein (UBE1L) and/or ubiquitin-like proteins such as ISG15. Alternatively, agents of the present invention may inhibit the activity and/or expression of the deconjugase UBP43. Agents of the present invention are useful in preventing or treating cancerous or neoplastic as well as pre-cancerous or pre-neoplastic cells. UBE1L is widely expressed in diverse human tissues and tumor cell lines. UBE1L acts as the activating enzyme for the ubiquitin-like protein ISG15. As demonstrated herein, UBE1L is a RA inducible gene target in acute promyelocytic leukemia and U937 and THP1 cells, implicating a broad biological role for UBE1L. Expression of ISG15 as well as the deconjugase UBP43 have also been found to be induced by RA and appear to be regulated in a coordinated fashion with UBE1L. As shown herein, this induction occurs only in RA sensitive cells, and not in RA insensitive cells. Further, coordinate regulation and physical association of UBE1L and ISG15 induces degradation of oncogenic proteins including, but not limited to, PML/RARα, cyclin D1, and PML, in RA sensitive cells. In addition, the induction of degradation of these oncogenic proteins by UBE1L and ISG15 appears to preferentially trigger apoptosis. PML/RARα degradation is inhibited by UBP43 transfection. Accordingly, agents which induce activity and/or expression of UBE1L or ubiquitin-like proteins such as ISG15 and agents which inhibit activity and/or expression of the deconjugase UBP43 are expected to be useful in treatment of neoplasia and pre-neoplasia, particularly RA sensitive cancers and cancers expressing oncogenic proteins such PML/RARα.

All-trans-retinoic acid (RA) treatment induces remissions in acute promyelocytic leukemia (APL) cases expressing the t(15;17) gene product, promyelocytic leukemia (PML)/retinoic acid receptors (RARα). Microarray analyses has revealed induction of UBE1L (ubiquitin-activating enzyme E1-like) after RA treatment of NB4 APL cells. The kinetics of this induction was studied in RA-sensitive NB4-S1 APL cells using a reverse transcription-PCR assay. UBE1L mRNA induction occurred by 3 hours after 10 μM RA treatment. These results were independently confirmed by Northern analysis and after 1 μM RA treatment by reverse transcription-PCR assay. In contrast, UBE1L expression was not induced during the same time period, despite 10 μM RA treatment of the RA-resistant NB4-R1 cell line. This is indicative of UBE1L being a direct retinoid target.

Further, the direct relationship between UBE1L induction and effective retinoid treatment of APL cells was demonstrated by examination of UBE1L immunoblot expression. For these experiments, immunogenic peptides described in detail in Example 5 were used to generate independent polyclonal antisera recognizing the amino or carboxyl termini of UBE1L protein, respectively. Chinese Hamster Ovary (CHO) cells that did not basally express UBE1L mRNA were transfected with a full-length UBE1L cDNA or an insertless vector. CHO cells transfected with UBE1L expressed UBE1L protein. In contrast, cells transfected with an insertless vector did not express this 112-kDa species. The UBE1L immunoblot expression profiles were also compared in RA-sensitive versus RA-resistant NB4 cells. UBE1L protein was basally expressed at low levels in both cell lines. However, protein expression was induced only after RA (1 μM) treatment of NB4-S1 APL cells.

A hallmark of RA response in APL is PML/RARα degradation (Yoshida et al. Cancer Res. 1996 56: 2945-2948; Raelson et al. Blood et al. 1996 88: 2826-2832; Nervi et al. Blood 1998 92: 2244-2251; Zhu et al. Proc. Natl. Acad. Sci USA 1999 96: 14807-14812). RA treatment has been reported to repress PML/RARα expression in NB4-S1, but not in RA-resistant NB4-R1 cells (Nason-Burchenal Differentiation 1997 61: 321-331). To examine the relationship in APL cells between UBE1L and PML/RARα expression, immunoblot expression profiles for these species were examined before and after 24 hour RA (1 μM) treatment of NB4-S1 cells. An inverse relationship was evident between UBE1L and PML/RARα expression both before and after RA treatment thus indicating a direct role for PML/RARα in regulating UBE1L expression.

A 1.3-kb fragment of the UBE1L promoter was then demonstrated to be capable of mediating transcriptional response to RA in a retinoid receptor-selective manner. PML/RARα, a repressor of RA target genes, abolished this UBE1L promoter activity. To examine the potential for PML/RARα to affect UBE1L, 1.3 kilobases (kb) of the UBE1L promoter upstream of the ATG translation start site was cloned into a luciferase-containing reporter plasmid. This reporter plasmid was transfected into CHO cells in the presence and absence of RA treatment. This fragment of the UBE1L promoter was capable of mediating transcriptional response to RA in a retinoid receptor-selective manner.

The relationship between PML/RARα and activity of this UBE1L reporter plasmid was examined when PML/RARα was cotransfected with this reporter plasmid. Cotransfection of PML/RARα with RARα led to a marked repression of UBE1L reporter activity before and after RA (1 μM) treatment. This inhibition depended on the dosage of transfected PML/RARα. In each experiment a cotransfected β-galactosidase reporter plasmid was used to control for transfection efficiencies. No appreciable effect of PML/RARα on the transcriptional activity of the β-galactosidase reporter plasmid was observed. Thus, PML/RARα repressed activity of this UBE1L reporter plasmid.

UBE1L has homology to E1. However, E1 mRNA was not induced after RA (1 μM) treatment of NB4-S1 cells, thus indicating different effects of RA on expression of UBE1L and E1.

A hallmark of RA response in APL is PML/RARα degradation. Accordingly, the ability of UBE1L as well as E1 to trigger PML/RARα degradation was next examined. Cotransfection assays were performed using cells that do not express PML/RARα. In these experiments CHO cells that did not express UBE1L and BEAS-2B cells that expressed low levels of UBE1L, but could be readily transfected with RARs or PML/RARα, were used. Degradation of transfected PML/RARα was triggered by UBE1L in a dose-dependent manner after transfection of CHO cells or BEAS-2B cells. This degradation of PML/RARα occurred in the absence of RA treatment. The PML domain of PML/RARα appears to be more sensitive to degradation by UBE1L than the RARα domain. Transfection of a truncated UBE1L (pSG5-UBE1L-T) did not cause PML/RARα degradation. To establish that PML/RARα degradation was a distinct UBE1L function, E1 was also transfected into BEAS-2B cells with PML/RARα. E1 did not cause PML/RARα degradation. Thus, transfection of UBE1L, but not E1, led to PML/RARα degradation even without RA treatment.

The effects of engineered overexpression of UBE1L in APL cells on growth or differentiation state of these cells was then examined. To overexpress UBE1L in APL cells, retroviral vectors (Ory et al. Proc. Natl. Acad. Sci. USA 1996 93: 11400-11406) were constructed to express UBE1L or no insert. Coexpressed GFP was used to enrich for retroviral-expressing cells after FACS sorting. HeLa cells, which do not express PML/RARα and basally express UBE1L at low levels, were used as a control for these experiments because retroviral transduction conditions were previously optimized in these cells. UBE1L overexpression was engineered independently in NB4-S1 and HeLa cells using the described retroviral transduction method. As a control, an insertless control vector was independently introduced into these cell lines as confirmed by immunoblot analysis. A striking difference in biological effects was observed after transduction of UBE1L into NB4-S1 versus HeLa cells. UBE1L overexpression in NB4-S1 cells resulted in the rapid induction of apoptosis as measured by the Hoechst staining of transduced cells. Three independent fields were examined for the insertless control NB4-S1 transfectants and 5.1% of these cells were apoptotic. Analysis of UBE1L-transduced NB4-S1 cells revealed a high proportion (39.7%) of apoptotic cells. These transductants did not exhibit morphological evidence of leukemic cell maturation. This lack of induced differentiation was confirmed by the absence of NET-positive cells (see Table 1).

Table 1: NBT Maturation Assays Performed on NB4-S1 APL Cells After 5 Days Treatment with RA (1 μM) [Designated +RA} or Vehicle (DMSO [Designated —RA] or Transduction of the UBE1L Retrovirus, Designated +UBE1L] as Compared to Transduction of the Same Retrovirus Without an Insert (Designated −UBE1L]

| Cell line | NBT, % |
|---|---|
| NB4-S1 (−RA) | 0 |
| NB4-S1 (+RA) | 92 |
| NB4-S1 (−UBE1L) | 0 |
| NB4-Sl (+UBE1L) | 0 |

Promotion of apoptosis was not observed in HeLa cells transduced with either the UBE1L or insertless retroviral vector. Thus, UBE1L transduction preferentially triggered apoptosis in PML/RARα-expressing cells. Induction of apoptosis was so rapid, however, that examination of the mechanisms signaling apoptosis was precluded.

As demonstrated herein, there is a tight link between UBE1L induction and PML/RARα degradation. More specifically, experiments described herein are demonstrative of an antagonistic relationship between UBE1L and PML/RARα. Further, increases in expression of UBE1L rapidly induce apoptosis in cells expressing PML/RARα. Accordingly, UBE1L is believed to be a target for repression by PML/RARα and induction of apoptosis in cells expressing this oncogenic protein.

Additional experiments in BEAS-2B human bronchial epithelial cells confirmed that co-transfection of UBE1L with transfected cyclin D1 triggers degradation of the oncogenic protein cyclin D1. Thus, the degradation program described herein is active beyond leukemia. In this study β-actin was used as a control to confirm that similar amounts of total protein were added per lane. Prior work has implicated cyclin D1 degradation as a chemopreventive target (Langenfeld et al. Proc. Natl. Acad. Sci. USA 94: 12070-12074; Boyle et al. J. Natl Cancer Inst. 1999 91: 373-379). Thus, these experiments directly implicate UBE1L in cancer chemoprevention. The deconjugase UBP43 is also implicated in the described degradation program in that transfection of UBP43 stabilizes cyclin D1 despite transfection of UDE1L in BEAS-2B human bronchial epithelial cells.

RA also augments the ubiquitin-like protein ISG15 expression in RA sensitive but not resistant NB4 cells. In addition, RA-treatment increases intracellular ISG15 conjugation in retinoid-sensitive NB4 cells. This is indicative of a link between increased ISG15 and UBE1L expression and induction of myeloid differentiation. Consistent with this is that RA-treatment increases intracellular ISG15 conjugation in retinoid-sensitive NB4 cells. A physical interaction between UBE1L and ISG15 was established in vivo using a transient co-transfection assay. This interaction was not observed when mutant ISG15 lacking essential c-terminal glycines was examined. Thus, these experiments are indicative of a coordinate regulation of UBE1L and ISG15. Accordingly, it is believed that, like UBE1L, induction of ISG15 can also enhance degradation of oncogenic proteins such as PML/RARα and promote apoptosis of cancer cells expressing these oncogenic proteins.

Agents that selectively induce UBE1L and/or ISG15 expression and/or activity in APL are thus expected to cause antileukemic effects by triggering PML/RARα degradation and apoptosis. Based on findings presented here and previous reports (Yuan, W. and Krug, R. M. EMBO 2001 20: 362-371), UBE1L is also expected to have an important biological role beyond APL. UBE1L maps to chromosome 3p, a region frequently deleted in lung cancers; UBE1L repression is frequent in lung cancers (Kok et al. Proc. Natl. Acad. Sci. USA 1993 90: 6071-6075; Carritt et al. Cancer Res. 1992 52: 1536-1541) where it may exert a tumor suppressive effect. This possibility, when coupled with the expression pattern of UBE1L in human tissues or tumor cells (McLaughlin et al. Int. J. Cancer 2000 85: 871-876) and results of UBE1L retroviral transduction reported herein, indicate that UBE1L regulates growth of both normal and neoplastic or pre-neoplastic cells.

PML/RARα degradation is also inhibited by UBP43 transfection as confirmed by co-transfection experiments with PML/RARα and UBP43 where UBP43 was able to overcome the ability of UBE1L to trigger degradation of PML/RARα. Accordingly, agents that selectively inhibit the expression and/or activity of the deconjugase UBP43 are also expected to trigger PML/RARα degradation and apoptosis in cancer cells expressing PML/RARα.

Cancer cells, and in particular RA sensitive cancer cells or cancer cells expressing oncogenic proteins such as PML/RARα can be contacted with agents of the present invention that induce UBE1L and/or ISG15 expression and/or activity or inhibit the expression and/or activity of the deconjugase UBP43 to trigger degradation of oncogenic proteins such as PML/RARα and apoptosis in the cancer cells. Activation of this newly identified pathway is also expected to trigger degradation of other oncogenic proteins in non-leukemia cells, including other neoplastic or pre-neoplastic cells. In a preferred embodiment agents used selectively induce UBE1L and/or ISG15 activity and/or expression or selectively inhibit UBP43 activity and/or expression.

Compositions of the present invention comprising an agent which induces activity and/or expression of UBE1L or ISG15 or inhibits UBP43 expression and/or activity can be administered to a patient to prevent or treat cancer, and in particular cancers such as APL which express the oncogenic protein PML/RARα. In a preferred embodiment, the agent selectively induces activity and/or expression of UBE1L or ISG15 or selectively inhibits UBP43 as selectivity of these agents should alleviate undesirable clinical toxicities that complicate RA or arsenic trioxide treatments. Compositions of the present invention preferably further comprise a pharmaceutically acceptable vehicle selected routinely by those of skill in the art based upon the type of cancer being treated and the route of administration best suited for treatment of that type of cancer. Effective amounts of the agent to be administered can be determined routinely by those of skill in the art based upon in vitro and in vivo assays demonstrative of pharmacological activity such as those described herein.

Agents for use in the compositions of the present invention which induce expression and/or activity of UBE1L and/or ubiquitin-like proteins such as ISG15 or inhibit the deconjugase UBP43 can be identified routinely by those of skill in the art in accordance using in vitro assays such as described herein. For example, test agents can be screened for their ability to induce UBE1L expression via reverse transcription-PCR as described herein which measures increases in UBE1L mRNA or immunoblot expression as described herein which measures UBE1L protein in the presence and absence of a test agents. An increase in UBE1L mRNA or protein levels in the presence of the test agent as compared to UBE1L mRNA or protein levels in the absence of the test agents is indicative of the test agent inducing UBE1L and being potentially useful as an anticancer agent. Similar assays can be performed to identify agents which induce ubiquitin-like proteins such as ISG15. Agents which inhibit the deconjugase UBP43 can also be identified routinely. Such agents can then be administered to prevent and treat cancer.

The following nonlimiting examples are provided to further illustrate the present invention.

### EXAMPLES

### Example 1

### Cell Culture and Induction Protocol

RA and dimethyl sulfoxide (DMSO) were purchased from Sigma Chemical Company (St. Louis, Mo.). Stock RA (10 mM) solutions were dissolved in DMSO, stored in liquid nitrogen, and used in the dark during experiments. RPMI 1640 and D-MEM were purchased from Cellgro/Mediatech (Herndon, Va.). The NB4 APL cell line expresses PML/RARα (Lanotte et al. Blood 1991 77: 1080-1086). NB4-S1 and NB4-R1 are RA-sensitive and RA-resistant clones of NB4 cells, respectively (Nason-Burchanel et al. Differentiation 1997 61: 321-331). These cells were cultured in RPMI 1640 media supplemented with 10% FBS as described by Nason-Burchenal et al. (Differentiation 1997 61: 321-331). Chinese hamster ovary (CHO) cells were cultured in D-MEM supplemented with 5% FBS, 100 units/ml penicillin, 100 units/ml streptomycin, and 2 mM L-glutamine in a 5% CO₂ humidified incubator at 37° C. Human bronchial epithelial cells (BEAS-2B) were cultured in serum-free LHC-9 medium (Biofluids, Rockville, Md.) in accordance with established techniques (Langenfeld et al. Proc. Natl. Acad. Sci. USA 94: 12070-12074; Boyle et al. J. Natl Cancer Inst. 1999 91: 373-379). HeLa cells were cultured in DMEM supplemented with 10% FBS, 100 units/ml penicillin, 100 units/ml streptomycin, and 2 mM L-glutamine in a 5% CO₂, humidified incubator at 37° C.

### Example 2

### Differentiation and Apoptosis Markers

NB4 cell differentiation was scored by using the nitroblue tetrazolium (NBT) reduction assay (Nason-Burchenal et al. Differentiation 1997 61: 321-331; Nason-Burchenal et al. Blood 1998 92: 1758-1767; Nason-Burchanel et al. Oncogene 1998 17: 1759-1768) Transductants were identified by green fluorescent protein (GFP) coexpression. Apoptosis was scored by using established techniques and Hoechst staining of transductants that co-expressed GFP (Nason-Burchenal et al. Blood 1998 92: 1758-1767; Nason-Burchenal et al. Oncogene 1998 17: 1759-1768; Stadheim et al. Cancer Res. 2001 61: 1533-1540). Digital images were collected by using an Olympus 1X70 inverted microscope, a cooled charge-coupled device camera, and a MiraCal Pro Single Cell Imaging System (Olympus LSR Research, Melville, N.Y.).

### Example 3

### Plasmid Constructs

A full length UBE1L cDNA containing plasmid was obtained in accordance with the method of Kote et al. (Gene Expression 1995 4: 163-175). The pGEM-HA-1E1 plasmid was obtained in accordance with the method of Handley et al. (Proc. Natl. Acad. Sci. USA 1991 88: 250-262). pSG5-HA-1E1 was constructed by cloning the HA-1E1 fragmented into the pSG5 expression vector. An EcoR1 fragment containing the UBE1L cDNA was cloned into EcoR1-restricted pSG5 to yield the pSG5-UBE1L plasmid. A truncated UBE1L plasmid (UBE1L-T) lacks an EclXI/SnaBI fragment in the carboxy terminus of UBE1L. The hemagglutinin (HA)-tagged PML/RARα expression vector was constructed from pCMX-PML/RARα and pCMV-HA (CLONTECH) plasmids. The pGL3-UBE1L Luc reporter plasmid contained the luciferase gene and 5′ promoter elements of UBE1L. It was constructed by using a PCR amplified fragment of the UBE1L promoter derived from NB4-S1 genomic DNA (forward primer: 5′-GCAACCGAGTGAGACTGTCT-3′ (SEQ ID NO:1); reverse primer 5′-GCGCTCAGAGATAGGGTTT-3′ (SEQ ID NO:2)). DNA sequence analysis confirmed this cloning.

### Example 4

### UBE1L mRNA Expression Assays

UBE1L mRNA expression was assessed by a reverse transcription-PCR assay in accordance with established methods (Kakizuka et al. Cell 1991 68: 663-674). The forward primer was 5′-AGGTGGCCAAGAACTTGGTT-3′ (SEQ ID NO:3), and the reverse primer was 5′CACCACCTGGAAGTCCAACA-3′ (SEQ ID NO:4). The PCR product was visualized by probing with a ³²P-labeled primer. Results were confirmed independently by Northern analysis using a 1.0-kb EcoR1/NcoI-radiolabeled UBE1L probe in accordance with standard techniques (Langenfeld et al. Proc. Natl. Acad. Sci. USA 1997 94: 12070-12074). This probe had limited homology to El.

### Example 5

### Generation of Anti-UBE1L Antisera

Two rabbit polyclonal antibodies against UBE1L were independently derived (Covance Research Products, Denver, Pa.) using one peptide within the amino terminus (DCDPRSIHVREDGSLEIGD (SEQ ID NO:5)) and a second peptide within the carboxyl terminus (PGSQDWTALRELLKLL (SEQ ID NO:6)). Specificities of these antisera were confirmed by immunoblot analyses of UBE1L-transfected CHO cells.

### Example 6

### Immunoblot Analysis

Immunoblot analyses were performed using established techniques (Langenfeld et al. Proc. Natl. Acad. Sci. USA 1997 94: 12070-12074; Spinella et al. J. Biol. Chem. 1999 274: 22013-22018). Anti-RARα antibody was provided by and can be purchased from P. Chambon (Institut National de la Santé et de la Recherche Médicale, Strasbourg, France) to detect PML/RARα (Nason-Burchenal et al. Blood 1998 92: 1758-1767; Nason-Burchenal et al. Oncogene 1998 17: 1759-1768). An anti-HA mAb was purchased (Babco, Richmond, Calif.) as was an anti-actin polyclonal antibody, C-11 (Santa Cruz Biotechnology, Santa Cruz, Calif.).

### Example 7

### Transfection Procedure

Transient transfection of BEAS-2B or CHO cells was accomplished by using Effectene and transfection methods in accordance with the manufacturer's instructions (Qiagen, Valencia, Calif.). A β-galactosidase reporter plasmid (pCH111) was cotransfected to control for transfection efficiencies.

### Example 8

### Retroviral Constructs and Transduction Procedures

MSCV-IRES-GFP was constructed to express UBE1L cDNA by cloning an EcoR1 fragment from pSG5-UBE1L into an EcoR1 site of this retroviral vector. Restriction endonuclease and partial DNA sequence analyses confirmed cloning was in the desired orientation. A vector without an insert served as a control. For each vector, 10 μg was transiently transfected using calcium phosphate precipitation along with the CellPhect Transfection kit (Amersham Pharmacia). The 293GPG packaging cell line was provided by R. Mulligan (Harvard University, Cambridge Mass.) and is available to other investigators. Forty-eight hours later viral supernatant from 293GPG transfectants (Ory et al. Proc. Natl. Acad. Sci. USA 93: 11400-11406) was used to transduce NB4-S1 or HeLa cells in the presence of 6 μg/ml polybrene (Sigma Chemical Company, St. Louis, Mo.). Twenty-four hours later, FACS analysis was performed, and cells positive for GFP expression were harvested by sorting and used for these experiments.

1. A composition for prevention or treatment of cancer comprising an agent which induces UBE1L or a ubiquitin-like protein ISG15 or an agent which inhibits the deconjugase UBP43.

2. The composition of claim 1 wherein the agent selectively induces UBE1L or a ubiquitin-like protein ISG15 or selectively inhibits the deconjugase UBP43.

3. A method for enhancing pro-apoptotic and degradative pathways of neoplastic or pre-neoplastic cells comprising contacting cells with the composition of claim 1.

4. A method for enhancing pro-apoptotic and degradative pathways-of-neoplastic or pre-neoplastic cells comprising contacting cells with the composition of claim 2.

5. A method for preventing or treating cancer in a patient comprising administering to a patient the composition of claim 1.

6. A method for preventing or treating cancer in a patient comprising administering to a patient the composition of claim 2.

7. A method for identifying agents for use in preventing or treating cancer comprising determining an agent's ability to induce UBE1L or a ubiquitin-like protein ISG15 or to inhibit a deconjugase UBP43.


SEQUENCE LISTING (6 sequences)

SEQ ID NO: 1 - DNA, 20 bp, Artificial Sequence
  Synthetic
gcaaccgagtgagactgtct

SEQ ID NO: 2 - DNA, 19 bp, Artificial Sequence
  Synthetic
gcgctcagagatagggttt

SEQ ID NO: 3 - DNA, 20 bp, Artificial Sequence
  Synthetic
aggtggccaagaacttggtt

SEQ ID NO: 4 - DNA, 20 bp, Artificial Sequence
  Synthetic
caccacctggaagtccaaca

SEQ ID NO: 5 - protein, 19 aa, Artificial Sequence
  Synthetic
DCDPRSIHVREDGSLEIGD

SEQ ID NO: 6 - protein, 16 aa, Artificial Sequence
  Synthetic
PGSQDWTALRELLKLL